(19) United States
(12) Patent Application Publication
(10) Pub. No.: US 2021/0100671 A1
(43) Pub. Date: Apr. 8, 2021

(54) Flexible Delivery System and Implantable Stent for Surgical Use

(71) Applicant: STENTORIUM LIMITED, Galway (IE)

(72) Inventor: Ardle Tomas McDonough, Galway (IE)

(21) Appl. No.: 15/733,497

(22) PCT Filed: Feb. 12, 2019
(86) PCT No.: PCT/IB2019/000138

§ 371 Date: Aug. 11, 2020

Related U.S. Application Data
(60) Provisional application No. 62/804,125, filed on Feb. 11, 2019.
(60) Provisional application No. 62/629,687, filed on Feb. 12, 2018.

(51) Int. Cl. A61F 2/962; A61F 2/89

(52) U.S. Cl. CPC A61F 2/962; A61F 2002/825; A61F 2/89

(57) ABSTRACT

The invention is a tube, which would be used to facilitate the movement of an object through the lumen of the tube or over the outside of the tube. The invention could also be used as a stent designed for indwelling in a body, where its purpose would be to assist in the drainage of liquid from one part of the body to another. The expected use of the tube would be in minimally invasive surgical procedures where greater flexibility, radial strength and pushability than that provided by prior art are desirable to improve the speed of operations and the success rate of operations; to reduce the level of trauma caused to patients and to improve patient recovery rate. It could have use in applications requiring an exceptionally flexible and highly pushable tubing such as invasive surgery in vessels that are or have become tortuous and narrow, such as veterinary procedures for small animals, paediatric ureteral conditions, angioplasty for certain conditions and certain neurovascular work.

### TECHNICAL FIELD

The technical field of this invention is biomedical tubing, including catheter, hypotube and stent uses. This invention is particularly suitable for use in procedures involving tortuous or narrow vessels or both.

### BACKGROUND OF THE INVENTION

The use of minimally invasive techniques in surgery has increased substantially over the past twenty years. This invention seeks to improve on flexibility, kink-resistance and pushability compared to the prior art.

The prior art in terms of currently available catheters, hypotubes and stents can work well in straighter vessels, where flexibility and kink-resistance are not immediate concerns. Coils and braids are commonly used in the walls of such tubing. In other cases, a polymer tube is used without any reinforcement needed.

In larger vessels, pushability can be obtained by using thicker wires in braids or coils or greater wall thicknesses than might be possible in narrow vessels.

However, when vessels are narrow, wall thicknesses and materials used within the tube are reduced to such an extent that the tube can be very fragile and prone to kinking, even upon handling prior to placement. The products normally used in this category are microcatheters, hypotubes and certain types of stent.

It is proposed that this invention performs substantially better than the prior art for these given conditions of narrow or tortuous vessels, such that theatre time is reduced with a reduction in products failing; post-operative mortality rates are improved and there is less trauma to vessels reducing recovery time and the need for subsequent operations.

Radial strength and pushability are related issues in the field of microcatheters. With wall thicknesses as low as 0.001″-0.004″, metal wires used in a braid or coil that is contained within a polymer wall are relatively weak in terms of providing radial strength and pushability and are surrounded by a minimal amount of polymer coating. Both the braid and coil can give a spring effect that is not desirable in terms of pushing into a vessel or bypassing an obstruction. Hypotubes give the radial strength and pushability needed to bypass obstructions, but metal tubes have a more limited bend radius. Laser-cut patterns, such as a coiled, interrupted-cut or a C-cut pattern, can significantly improve the bend radius of a hypotube. Coil-type patterns are prone to unravelling into a coil when pushed to the limits of flexibility. The C-cut and interrupted-cut patterns perform better in this regard, but there is a need for greater flexibility than they provide to reduce the force needed to push the tube and thus reduce possible trauma to the vessel. Greater flexibility can only be obtained in C-cut or interrupted-cut patterns by increasing the size or amount of the cuts or by changing the layout of the cuts. The limiting factor is that this makes tears or kinks more likely to occur. The invention described here was designed with these factors in mind.

This invention combines the flexibility and pushability of a guidewire with the radial strength of a hoop by attaching a wire or a number of wires to a series of hoops. A guidewire is generally a solid wire surrounded by an outer material to make the guidewire more atraumatic or lubricious. It is used as the primary device to pass through a vessel and create a path along which a tube may be passed. Solid wires have a much greater flexibility and kink resistance than tubes. As with a guidewire, use of superelastic Nitinol wire would increase flexibility of the tube.

A solid hoop has the same kink-resistance as a solid tube, being a short tube. However, kinking forces from a bend in a vessel do not act upon a short hoop in the same way that they act upon a longer tube that has to curve around the bend. The proposal here is that a series of hoops is more flexible than a laser-cut hypotube, since it has more free space between the hoops than a hypotube has within its cuts. The principal relevant prior art is the laser-cut, metal hypotube.

Plastic tubing is cheaper to produce and would be used in any instance in which it could be used effectively. Plastic tubing kinks easily and has low radial strength, but if it can serve a biomedical purpose effectively, it will be used. There is no merit in comparing the tubing proposed in this invention with plastic tubing, since it would only have commercial uses where plastic tubing does not function sufficiently well. To that extent, it is effectively a different product with different uses and the plastic tubing or plastic stent prior art is not relevant.

The general purpose of the invention is to provide for faster and easier placement of stents in tortuous conditions, while minimizing trauma due to enhanced flexibility and kink-resistance.

### BRIEF SUMMARY OF THE INVENTION

The unique design of these tube iterations allows for the creation of significantly more flexible and more kink-resistant tubes than the prior art, with the added possibility of a 1:1 torque response and excellent pushability of a guidewire in the same device. This invention would be designed for use as delivery devices, stents or other biomedical usage. The hypotube iteration should be more flexible than predicate hypotubes; the catheter or microcatheter iteration should have more radial strength and more pushability than predicate catheters and the tube, when used as a stent, should have greater kink resistance and radial strength than the prior art. The prior art in stents generally has sufficient kink resistance and radial strength for the intended function, so it is unlikely that this invention would be used as a stent, but there may be some application as such.

There are structural differences between the tube design of the instant application and the tube designs found in the prior art, leading to functional differences that allow for the innovative tube of the instant application to function in very narrow and tortuous vessels as a stent or as a delivery device or for different applications.

Certain iterations of the tube design in this invention are of relatively high compressive strength, while others are of lower compressive strength. Likewise, some iterations possess high tensile strength and others, less so. The main defining factors are the use or lack of use of one or more than one wire attached to hoops and how closely connecting those hoops are.

There are a multitude of ways to cut a tube into metal hoops and to render the hoops free of burrs and smooth, for example, mechanical cutting, laser cutting, etching or molding and various cleaning processes. The most efficient method of manufacture and assembly will be worked out by trial and error. Assembly could be made easier by making the bottom of the tube slightly heavier than the top, such that in a low friction setting, the top of each hoop will be aligned for insertion of a wire. Alternatively, the wire could be placed in a given iteration before the tube is cut, if the hoops in that iteration are cut in less than 360° sections. There is a wide variety of methods of manufacture and it is likely that these will improve over time.

This invention can be used for very narrow and tortuous anatomy, such as in pediatric and veterinary anatomy and in some parts of the adult human anatomy. An adult domestic cat ureter is approximately twenty thousandths of an inch in diameter. Delivery device dimension and functionality is an issue that is central to the design of a stent delivery system. for the cat ureter. Stents currently on the market and described in the prior art are incapable of being made to certain dimensions. The hypotube of the instant application can be designed to accommodate a wide range of sizes and dimensions thus addressing a long felt need in the art of delivery device and stent design.

Kinking forces present in vessels can cause stent or catheter or delivery device failure. This is particularly apparent when the vessel is highly tortuous. Tube design for these conditions, therefore, needs to be highly kink-resistant. Some of the iterations of this invention can be used as stents and other iterations would undergo the same stresses in use as delivery devices. Since the hoops making up the cylindrical body of the tube in this invention can open and separate in response to bending forces in tortuous anatomy, it is highly kink-resistant in this regard. The same bending forces exerted on a tube that cannot open and separate to the same extent, or that can only bend insofar as the material will allow in the case of solid polymer tubing, can cause kinking of the tube or unravelling of the tube in the case of highly flexible, spiral-cut tubes.

Radial compression is a problem with the use of microcatheters since the braids and coils used to form the tube wall are generally made from very thin wire in a polymer jacket to facilitate very narrow wall thickness of a few thousandths of an inch. The tube in this invention is generally made of metal hoops of a few thousandths of an inch in wall thickness. It is designed to withstand significantly higher external forces from both intrinsic and extrinsic strictures than a microcatheter. This gives greater protection and ease of movement to a stent or other device being passed through the lumen of the tube in this invention. The kinking that can occur in microcatheters from such forces is also much less likely to occur since the tube in this invention is generally made of metal hoops, which are much less likely to kink than a thin wire braid or coil.

Where the flexibility or pushability required for a given clinical use cannot be obtained from current hypotube laser-cut designs, this invention is of use. The principle of this invention is to introduce as much free space as possible into the body of the tube, while retaining as much pushability, radial strength and structural rigidity as is necessary for the hypotube to function as intended. Since the design can provide for a significantly greater combined pushability and/or greater flexibility than predicate hypotubes, the stent of the instant application can pass through tighter and more tortuous vessels than predicate hypotubes with less trauma to the vessel.

A general explanation of the hypotube design is that it would be a series of latitudinal hoops connected by a longitudinal wire or a number of longitudinal wires. This design would be useful for conditions where commonly available spiral-cut, C-cut or interrupted-cut hypotubes are not flexible enough, likely to unravel or not sufficiently kink-resistant for use. Its purpose would typically be to facilitate the passage of a catheter, guide wire, stent, camera or other device through the biological systems of a body or to act as a stent or catheter.

The principle of using hoops for certain iterations of the tube in this invention, as opposed to a coil or a braid, is that they would have greater radial strength, while retaining the same or greater flexibility depending on the amount of space between hoops. Care should be taken to avoid having more space between hoops than would be needed to keep an obstruction from impacting on the lumen of the tube. If using fully separated hoops, the maximum total gap between the hoops should not exceed the minimum point or smallest side of an obstruction to ensure the lumen of the tube is not impacted by the obstruction. Additional radial strength has an impact within vessels, providing for safer placement where fragile braids or coils can be easily kinked radially or through excess compressive force from pushing. Like a coil, hoops will press together on the inside of a bend and separate on the outside of a bend, leading to relatively easy passage around bends. There is not, however, the same spring effect as with a coil upon encountering or leaving a tightly-fitting dimension, which is advantageous in preventing trauma to the vessel.

There can be a wide variety in the kerf widths or cut widths used to form hoops to suit the clinical application. There can also be a wide degree of variance in how complete the cuts are through the tube to facilitate clinical usage, from completely cut hoops attached to a wire to partially-cut hoops that remain part of a tube.

The easiest method of manufacture for this hypotube design might be to laser weld a wire or rod to a hypotube and laser-cut the hypotube into a series of latitudinal hoops. Further, wires could be laser-welded to the hypotube then for structural rigidity. An issue to be overcome by the design proposed here is that if using laser-welding, the laser-weld strength should be sufficiently strong to hold the longitudinal wires to the latitudinal hoops to a point where the kink resistance of the tube as a unit approaches that of solid wire. A more cost-effective and possibly more effective alternative to welding might be to produce a tube with one or more pre-formed lumen(s) on the outer diameter of the tube or in an outer jacket encasing the tube, into which a wire is placed after the tube is laser cut into a series of latitudinal hoops. In this iteration, the wire and hoops are independent of each other and not touching directly, but they act in unison.

In the manner listed in the paragraph above, a hypotube is created that has the pushability of solid wire, the kink resistance of solid wire, the radial strength of complete metal hoops and greatly increased flexibility in comparison with the most flexible hypotubes presently available, which would be spiral-cut or interrupted-cut designs. As spiral-cut designs move towards maximum flexibility, they approach the properties of a coil and lose pushability and they can uncoil also. As C-cut and interrupted-cut hypotubes move towards their maximum flexibility, kinking occurs as the cuts in the hypotube cannot take the strain imposed by bends. The design proposed above should overcome these issues.

This hypotube could be made from a variety of metals, but the longitudinal wires used should be made of a flexible, superelastic metal such as binary Nitinol since these wires dictate the flexibility of the hypotube and are also part of its resistance to kinking. The tube used to make the latitudinal hoops could be made of a cheaper metal, such as Stainless Steel 304 or 316, since these exhibit somewhat similar properties to binary Nitinol in tubing of this nature. Stainless steel is cheaper than Nitinol and it is easier and therefore cheaper to clean after laser-cutting. If, however, using this hypotube design as a stent, for short or longer term implantation in a body, Nitinol tubing should be used for biocompatibility. Laser welding is possible from Nitinol to itself but is not generally possible between Nitinol and stainless steel, so this would need to be factored into any design and choice of tube material.

There are a vast multitude of ways in which this tube can be created, but the common factors will generally be a wire or rod made from any material extending longitudinally down the length of the tube, such that pushability of the tube is generated through the wire, and radial reinforcement. The wire would generally be either embedded in the wall of the tube or attached to a coil or a series of hoops or a braid in the inner lumen of the tube. The lumen of the tube can be made as kink resistant as is necessary with metal hoops or with a braid or coil or none of these, if kink resistance is not paramount to the function of the tube. It may be desirable to attach the wire to the hoops or coil or braid or a combination of these, but it may also be more cost-effective to use multiple lumens where the wire and hoops or coil or braid are not connected or where they are not used, and a wire is placed into the wall of a tube.

An iteration where pushability is less important than flexibility, or being as atraumatic as possible, would be the use of hoops without a wire or rod connected to them. These hoops could be connected to each other by a coil or possibly by a spiral-cut section of tubing or they could be left unconnected within a polymer jacket, such that some sort of pushing force would be needed to advance the hoops and the tube unless the pushability of the polymer jacket was sufficient in itself. This may suit in certain neurological uses, where maximum flexibility and minimum possible trauma may be needed in the device. Anti-migration meshes or coils would be needed also, in a low profile state while advancing the device and expanded for deployment of the device.

A hypotube is typically composed of metal for the most part, but one iteration of the tube in this invention could be a polymer tube with a longitudinal rod or wire inserted into the wall of the tube and radial reinforcement such as metal hoops, a metal coil or a metal braid. The purpose of the longitudinal wire would be to add pushability and act as a guidewire within the tube. Several longitudinal wires within the wall of a polymer tube provide radial strength and pushability, if desirable in a catheter tube or a polymer stent tube.

Where the metal iteration would be generally a form of hypotube, assuming use in narrow vessels; a polymer iteration could be a form of catheter or microcatheter in biomedical usage. Either could also be used as a stent with standard additions such as anti-migration designs, for example, pigtails at either end or spiral cup-shaped ends.

It would be possible to increase flexibility at any given point by varying the pattern of wires placed longitudinally in the wall of the tube to introduce gaps on one or other side. Pushability would be affected by these changes, so there is a trade-off if extreme flexibility is needed.

It may also be advantageous to allow for movement of the wires in and out of the tube to bypass a given stricture or tortuous bend or obstruction in the vessel. For example, if wires are placed at 0°, 90°, 180° and 270°, it should be possible to turn the tube by pulling wires out and pushing other wires, perhaps using a handle designed for this purpose. This type of maneuvering is similar to, but more advanced than, using a guidewire and catheter in tandem, pushing the guidewire where necessary and withdrawing the guidewire where the catheter passes freely through the vessel to reduce the possibility of trauma to the vessel.

Gaps between hoops are obviously weak points in terms of radial strength of the tube. The point to note is that the object of the placement of the tube is to maintain a clear path for liquid, passage of a stent or other device. It does not matter if the tube has weak points, so long as those weak points do not impact on the maintenance of a clear path throughout the vessel. The tube must be capable of being placed in the vessel without any collapsing of the hoop structure and without any stricture or obstruction impacting on the lumen of the tube. The reason for the various iterations of this invention is that these two essential requirements may necessitate different solutions for different conditions or different vessel structures. For example, a very delicate vessel with a stricture that is easily pushed apart may be opened by a tube with latitudinal hoops and without any longitudinal wire, thus reducing the pushing force exerted by the tube on the vessel.

The latitudinal hoops should be made from metal, where radial strength is needed, since metal can provide good radial strength from a very thin wall thickness. Metal hoops would provide much greater radial strength than an equivalent polymer wall thickness. This means that the tubes in this invention can be effective in much narrower wall thicknesses than a polymer tube. This has advantages where vessels are very narrow, such as in pediatric or neurological usage. The metal hoop section, possessing high radial strength and a proportionally large lumen, can be used to deliver stents, guidewires, scopes and other medical devices in narrow, tortuous vessels.

It would be desirable to use inner liners or an outer jacket for passage of devices through the tube or for passage of the tube through a vessel. This would add to the wall thickness of the tube, but it would be preferable if the vessel can accommodate the additional wall thickness. Liners or jackets can be as thin as 0.001 inches, which should generally be possible to accommodate.

It may be desirable to use an expandable hoop structure or coil or braid or mesh, such that the tube can be inserted in an unexpanded state and subsequently expanded, leading to a reduction in possible trauma to the vessel and an increase in the speed of placement of the tube.

The use of a longitudinal wire proposed in this invention may improve certain prior art stent design. A stent using a coil with a longitudinal wire attached to end caps connected to either end of the coil, would be improved by attaching the wire to the coil itself or close to it, since the attachment of a wire to the interior wall or exterior wall decreases the bend radius of the tube beyond that of a tube with a wire connected to either end through the center of the tube. The bend radius will depend on the diameter of the wire and the physical properties of the tube or coil, but in small dimensions, there should generally be a positive impact, whereby the bend radius is decreased by the tube design proposed in this invention and the tube becomes more flexible.

A flat wire may be more suitable to use than a circular wire because it would have more surface area close to the tube than the curved surface of circular wire. Similarly, a wire that is more oval than flat or round could be used to benefit from the flexibility of circular wire and the attachability of flat wire. It may also be advantageous to use a tube that is not perfectly round or that is flatter at certain parts of its circumference.

This invention should ensure that a minimum of trauma is caused to vessels where flexibility beyond the physical characteristics of currently available spiral-cut and interrupted-cut hypotubes is necessary or desirable. It may also have uses in a wide variety of surgical uses not described in this application, such as neurological applications, biliary applications, cardiovascular applications, etc.

Standard additions of inner liners, outer jackets, hydrophilic coatings, marker bands, etc. would be used to suit a given biomedical application.

It could also be possible to use a coil or braid instead of cutting hoops into a tube. This would reduce production costs since there would be no need to laser-cut the tube into hoops. Wires could be laser-welded to the coil in the same way as described above to give sufficient rigidity and pushability to the coil and to prevent a spring effect where a coil would bounce back on encountering an obstruction in its path or spring forward on passing an obstruction with too much compressive force exerted on it by the user pushing it from the proximal end. A coil without wires welded to it might have a further complication of not pulling easily past a placed stent regardless of the use of inner liners, which would be resolved by this invention.

Following on from the use of a coil as a tube, as described above, would be an application for use in a coil in the distal end or tip of a guidewire. Coils are used in guidewire tips for flexibility. To ensure that the coil stays attached to the guidewire, the guidewire generally extends to the end of the coil tip so that the coil is connected at both of its ends to the wire by gluing or welding. The flexibility of the tip is increased by grinding down the wire inside the coil and by lengthening the coil section. The method of attaching the wire in this invention to the interior wall or exterior wall of the coil will allow guidewire tips to be more flexible and more pushable. The wire should be gradually ground down from the center outwards, so that a portion of the outer diameter of the wire runs from proximal end to the distal end of the coil tip along the interior wall of the coil.

Another option would be to use the invention of hoops attached to a ground down wire in a guidewire tip. This would provide a greater flexibility than that of a standard coiled guidewire tip, since the wire would not be attached to the end of the coil through the center of its lumen, but rather along the inner surface of the hoops. A PTFE or similar material jacket could be placed over the tip to prevent materials entering into the tip and the final hoop should have no hole in it for the same reason. Furthermore, there is likely to be a myriad of uses for the tube structure described above in non-biomedical applications such as scientific work, instrumentation, electrical work, etc. Its properties of radial strength and pushability should have greater impact in very small dimensions, since these properties can be achieved by reinforcing larger dimension tube walls. The property of flexibility of this hypotube design may be of use in some larger dimension tubing. A further advantage would be that it uses less material in its manufacture than other forms of metal tubing. This benefit is outweighed at present by the cost of laser-cutting, but that may not always be the case as resources decline. Given that a larger dimension tube can have sufficient radial strength and pushability from using increased volumes of material in the longitudinal wires and latitudinal hoops, it may be that plastics could be used instead of metal for a larger dimension tube and that these could be cut by a cheaper means than laser-cutting, such as a mechanical blade. It could also be possible to use a more rigid type of plastic tubing in smaller dimensions, depending on the wall thickness and radial strength properties needed.

Fiber-optic cables would be another possible use for the tube designs proposed in this invention, since the bend radius can be decreased using these designs, resulting in decreased micro-bending and macro-bending losses and improved kink resistance.

It may be possible to place a tiny motor or a series of tiny motors in the lumen of a tube or in the wall of a tube to deliver it to a given location. This would allow for more flexible tubes to be used, since pushability would not be required to the same extent. Such motors have been developed and are currently intended for use in delivering drugs to given locations in the body. As they are further developed, use in minimally invasive surgery would seem to be a logical step.

The essential characteristics of the tube design are a combination of higher flexibility, higher pushability and higher kink resistance and resistance to breaking than other tube designs.

Where this combination is desirable, for example in tortuous conditions that may occur in small animal biology, human infant urology, bile duct, pancreas, human neurology and various smaller vessels, in delicate vessels or vessels with difficult strictures or obstructions, there should be an advantage to using this tube design over other tube designs. Furthermore, for certain conditions where pushability is necessary, it may be the case that the tube proposed in this invention could be advanced through the vessel without needing to be tracked along a guidewire. This would remove a step in an operation, reducing theatre time, which in turn lowers the cost of operations allowing for the treatment of more patients.

As such, it may be desirable to include standard guidewire distal end modifications in the wire inside the tube to increase the flexibility of the distal end of the tube, such as reducing the diameter of the wire or the use of coiling around a ground wire tip.

Materials most suitable for an outer jacket might be PTFE, FEP, fluoropolymer, silicone, polyurethane, polyethylene, PEBAX and nylon, but any material approved for use in medical device can be used.

The tube could be flared at the proximal end. The flared end can be attached to the connector cap or handle by gluing or by over-molding or by welding the proximal end on to a connector cap or handle. The distal end would have a radiopaque marker. The radiopaque marker would typically be a marker band, a radiopaque filler encompassed in the plastic of the sheath or radiopaque ink.

The tube could have markings on its length to denote distance of the tube within the body during a surgical procedure. This would be of benefit during stent or scope placement.

Other additions to the tube design could consist of holes created in the membrane or jacket of the tube to allow liquid to flow into or out of the tube, according to the pressure exerted. The holes could be created by perforating the membrane with a sharp tool or by laser or some other means.

The metal of the tube can be plated with a heavier metallic material, such as gold or tungsten, to enhance its radiopaque properties, prevent encrustation and to provide a smooth surface, onto which bacteria cannot easily adhere. Anti-bacterial or anti-encrustation coating may also be applied to the stent's surface to prevent or reduce encrustation of the stent for its indwell duration or electropolishing or other techniques can be used to smoothen the surface with the same objective. Alternatively, a marker band could be used to enhance radiopacity at the distal end or at other points of the body of the tube.

Another possible iteration would be the use of alternating or non-alternating extensions from a wire that are expandable and collapsible, mechanically or by shape memory or by dilation, for example by balloon. The extensions can be made of solid metal but would most likely be made of some form of braid or mesh. Essentially these extensions would form a tube in their high profile state and would cross over each other in their low profile state, such that the device can be easily tracked along a vessel. For visualization purposes, in the case of using pairs of these extensions attached to a single wire in either non-alternating (one opposite the other) format, the extensions would be like an incomplete hoop in their high profile state. It could be possible to use a complete braid or mesh attached to the longitudinal wire in a low profile state for rapid placement of a tube in high profile state to form a delivery device that is still highly flexible, albeit not as flexible as the incomplete hoop iteration. A complete braid tube could be harder to remove than the incomplete hoop version, depending on the force being exerted against the wall of the vessel in either case.

This iteration would have the pushability of a guidewire and the flexibility of a guidewire with lower radial strength than other iterations, which may suit more delicate and more tortuous vessels. It would also have the kink-resistance of a guidewire when tracking along the vessel, since the tube could generally not kink or break in its low-profile state. One important consideration would be that the extensions on either side of the device could not cross over each other in the low profile state to such an extent that the ends protrude out over the edge of the opposing side. This would cause trauma to the vessel. It could not be possible for the ends to protrude out over the edge of the opposing side in the low-profile state by bending force or other force either. This can be achieved by collapsing the opposing sides into each other, such that any possible force would only push the extensions further into each other, or by folding an extension into itself to be held within a sheath in its low profile state. Outer jackets, inner liners and other standard industry additions could also be used to improve the device.

It would not be essential to have the extensions in a consecutive row on a single wire. They could be attached to wires on opposing sides of a tube and expanded to their high profile state, such that a pair of extensions would be on the opposite side of the tube to the next pair of extensions. In this way, a highly flexible tube structure would be created that could be inserted in a low profile state without the need for the extensions to cross over each other. It would also not be essential for the wire to be attached directly to the mesh extensions. These could be contained in a polymer tube in a low profile state to be expanded to become a tube structure that is attached to the longitudinal wire or that incorporates the longitudinal wire in the tube structure in some other manner. Proximal and distal tube elements of this structure could be formed by having the longitudinal wire or longitudinal wires be connected at the distal end and the proximal end to or formed into anti-migration devices, such as an expandable mesh or coil. It would be possible to use more than two wires also with extensions on each wire. A multitude of wires with smaller extensions would mean that the radial strength of the tube would be generated by the wires as much as the extensions. This may result in a softer, more flexible stent than one which generates its radial strength from pushing outwards latitudinally, as in the case of a self-expanding metal stent, than a combination of longitudinal and latitudinal resistance to force. This iteration could be an effective stent as well as an effective delivery device.

### BRIEF DESCRIPTION OF THE DRAWINGS

FIG. 1 is an illustration of a cross-sectional view of one iteration of the hypotube;

FIG. 2 is an illustration of a longitudinal view of one iteration of the hypotube;

FIG. 3 is an illustration of a longitudinal view of a second iteration of the hypotube;

FIG. 4 is an illustration of a longitudinal view of a third iteration of the hypotube;

FIG. 5 is an illustration of a longitudinal view of a fourth iteration of the hypotube;

FIG. 6 is an illustration of a cross-sectional view of a fifth iteration of the hypotube;

FIG. 7 is an illustration of a longitudinal view of a fifth iteration of the hypotube;

FIG. 8 is an illustration of a cross-sectional view of a sixth iteration of the hypotube;

FIG. 9 is an illustration of a longitudinal view of a sixth iteration of the hypotube;

FIG. 10 is an illustration of a longitudinal view of a seventh iteration of the hypotube;

FIG. 11 is an illustration of a longitudinal view of an eighth iteration of the hypotube;

FIG. 12 is an illustration of a longitudinal view of a ninth iteration of the hypotube before a stricture;

FIG. 13 is an illustration of a longitudinal view of a ninth iteration of the hypotube passing a stricture;

FIG. 14 is an illustration of a longitudinal view of a tenth iteration of the hypotube;

FIG. 15 is an illustration of a longitudinal view of an eleventh iteration of the hypotube;

FIG. 16 is an illustration of a longitudinal view of a twelfth iteration of the hypotube;

FIG. 17 is an illustration of a longitudinal view of the distal end of a thirteenth iteration of the hypotube;

FIG. 18 is an illustration of a longitudinal view of the distal end of a fourteenth iteration of the hypotube;

FIG. 19 is an illustration of a longitudinal view of the distal end of a fifteenth iteration of the hypotube;

FIG. 20 is an illustration of a longitudinal view of a sixteenth iteration of the hypotube;

FIG. 21 is an illustration of a longitudinal view of a seventeenth iteration of the hypotube;

FIG. 22 is an illustration of a longitudinal view of an eighteenth version of the hypotube;

FIG. 23 is an illustration of a longitudinal view of a nineteenth version of the hypotube;

FIG. 24 is an illustration of a longitudinal view of a twentieth version of the hypotube;

FIG. 25 is an illustration of a longitudinal view of a twenty-first version of the hypotube; and

FIG. 26 is an illustration of a longitudinal view of a twenty-second version of the hypotube.

### DETAILED DESCRIPTION OF THE INVENTION

There are numerous possible embodiments associated with the tube and stent designs for the invention described herein and these are detailed in the claims below. All drawings, summaries, descriptions, embodiments and objects are intended to be illustrative rather than limiting.

### Embodiments

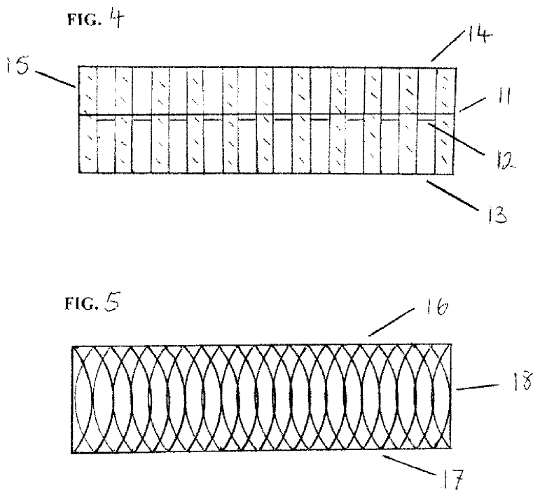

The wire attached to the hoops can consist of a metal or a plastic polymer material such as, but not restricted to PVC, polyurethane, polyethylene, silicone, FEP, PEBAX, polyamide, polyimide and PEEK. Alternatively, the wire can be a combination of a polymer tube surrounding a metal cannula or mandrel (FIG. 3A).

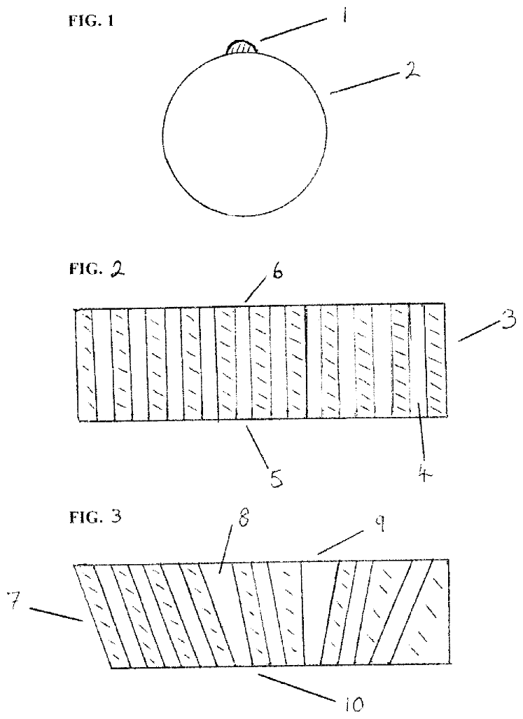

FIG. 1 shows a cross-sectional view of a hypotube. This hypotube would be made by laser-welding a wire 1 to a metal tube 2.

FIG. 2 shows a longitudinal view of a hypotube. The hypotube has been laser-cut into hoops 3 or as close to full hoops as is practical with cuts at 90° angle 4 across the width of the tube and wires 5, 6 attached longitudinally at opposite sides,

Designs could use cuts at a wide variety of other angles depending on the needs of the user. It may be preferable to reduce drag on the hoops and angle the laser cuts at 80° for example. FIG. 3 shows a longitudinal view of a design with a 70° cut 7. Similarly, it may be preferable to use non-uniform cuts 8. A cut could start at 0.5 mm kerf width at one side and finish at 1 mm kerf width for example, if it was desirable to have one side more flexible than the other for a portion or for the entire length of the hypotube. Alternating kerf widths could be used to move points of stress along the tube as would be used in interrupted-cut designs. The standard design for structural rigidity would be 90° uniform cuts with variances to the angle of the cut and the uniformity of the cuts to introduce additional flexibility for specific uses. Wires are attached longitudinally at 9 and 10.

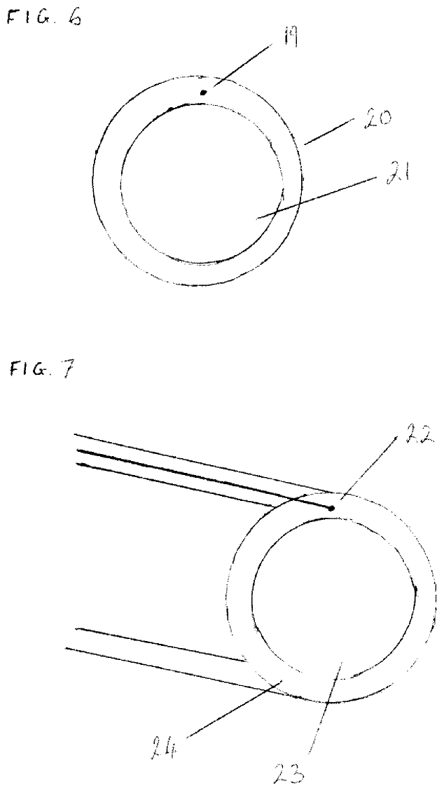

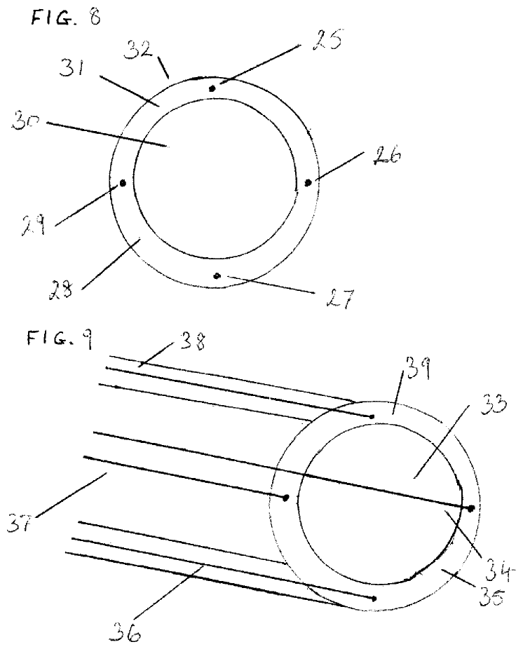

FIG. 4 shows a longitudinal view of a third iteration of the hypotube design with four wires 11, 12, 13 and 14 laser-welded to the latitudinal hoops 15 for additional structural rigidity.

FIG. 5 shows a longitudinal view of a fourth iteration of the hypotube design with wires 16 and 17 laser-welded to a coil 18.

FIG. 6 shows a cross-sectional view of a tube 20 with central lumen 21 and a wire 19 inserted into the wall of the tube. The tube would typically have radial reinforcement in the form of hoops or a braid or a coil. FIG. 7 is a longitudinal view of the same tube with the wire 22 inserted into the wall 24 of the tube with a central lumen 23.

In a slightly different iteration, FIG. 8 is a cross-sectional view of a tube 31 with a central lumen 29 and wires 25, 26, 27, 28 inserted into the wall 30 of the tube. Placement of the wires can be in any part of the wall of the tube and near the inner diameter or the outer diameter 32. FIG. 9 is a longitudinal view of the same tube 39 with a central lumen 33 and wires 34, 36, 37, 38 inserted into the wall 35 of the tube. The tube would typically have radial reinforcement in the form of hoops or a braid or a coil.

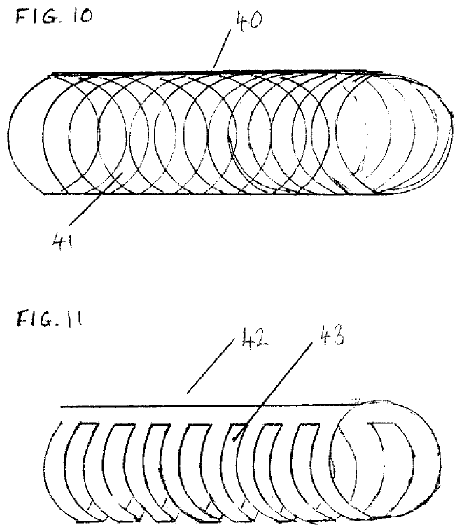

FIG. 10 is a longitudinal view of a tube or stent made from a coil or mesh or braid 41 with a wire 40 attached to the wall of the coil or mesh or braid. This wire could be attached to the inner wall or outer wall of the coil or mesh or braid.

FIG. 11 is a longitudinal view of a tube 42 with part of the tube cut latitudinally 43, such that the uncut part of the tube next to the cut section becomes a series of hoops.

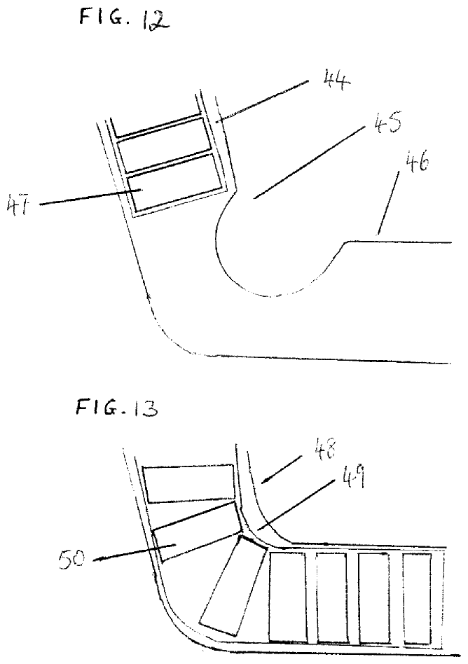

FIG. 12 shows a longitudinal view of a series of hoops 47 enclosed in an outer jacket 44 encountering a stricture 45 in a bodily vessel 46.

FIG. 13 shows the same series of hoops 50 enclosed in an outer jacket 49 pushing out the stricture by virtue of the radial strength of the hoops and the force pushing them with the hoops remaining closely connected on the inside of the bend and spreading outwards on the outside of the bend, such that a minimum of trauma, as a result of force or radial stress at any particular point, is caused to the walls of the vessel 48.

FIG. 14 is a longitudinal view of a tube made of hoops 52 connected by a coil or mesh or braid 51. It could be possible to make this tube from a single piece of metal by using a heavy spiral cut between the hoops if that provided sufficient flexibility for the clinical use. This tube could also have a wire attached to the outer or inner wall of the tube and could be used as a stent, as could many of these tube iterations.

FIG. 15 is a longitudinal view of a tube with metal hoops 56 inside the outer jacket 53 and an inner liner 55 inside the metal hoops. A wire or rod 54 is inserted between the metal hoops and the inner liner. This is a variation in the placement of the wire, which may be desirable for certain manufacturing or clinical needs.

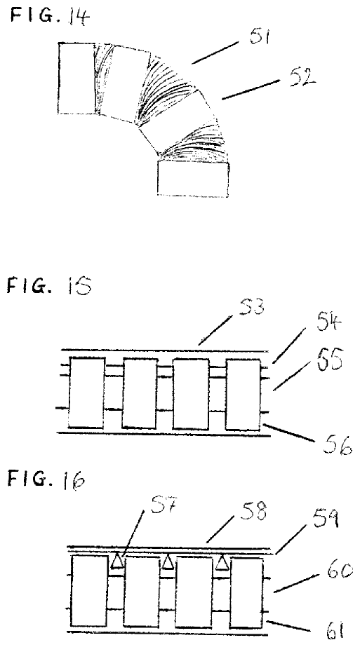

FIG. 16 is a longitudinal view of a tube made up of an outer jacket 58 with triangle parts 57 attached to a wire or rod 59 and with unconnected hoops 61 between the triangular parts and an inner liner 60 passing through the hoops. The triangle design would allow for movement of the hoops through a given angle in both directions as well as maintaining the hoops in position.

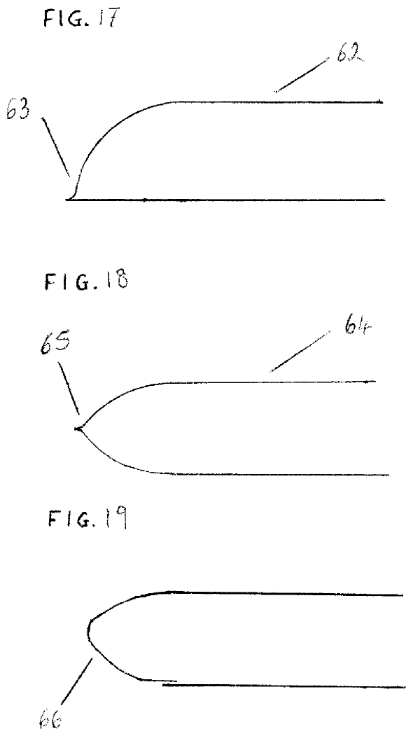

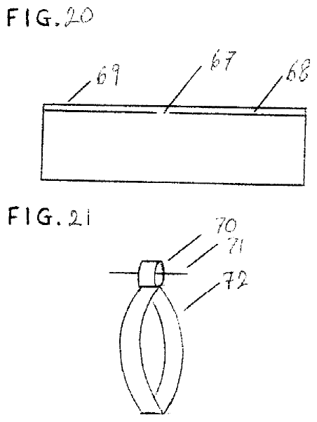

FIG. 17, FIG. 18 and FIG. 19 show the distal ends of tubes. In FIG. 17, the distal end 63 of the tube 62 is closed by bringing one side down to the other side. In FIG. 18, the distal end 65 of the tube 64 is closed by bringing the edge of the tube in to a central point. In FIG. 19, the distal end 66 of the tube is formed by running one side of the tube out to a central point and back inside the other side of the tube. It may work better to form these distal ends by using a wire inside the wall of the tube as in previous iterations described here, rather than just a polymer end. The intention of these designs is to reduce bodily materials catching on the distal end of the tube or building up inside the distal end of the tube. The force needed to open the distal end of the tube should not be so great as to impact on the atraumatic passage of a stent or endoscope or guidewire through it.

FIG. 20 shows a longitudinal view of a tube 69 with a wire or rod 68 in the wall of the tube and a gap 67 in the length of the wire. The principle of this is to facilitate higher levels of flexibility where necessary. There would be significantly reduced pushability of the distal section(s) of tubing after a gap or gaps in the wire had been passed into the body.

FIG. 21 shows a side view of a hoop 72 with a smaller hoop 70 attached to it and a wire or rod 71 passing through it. The smaller hoop would allow for movement of the larger hoop along the axis of the wire and at angles to it, while leaving the entire lumen of the larger hoop free for passage of stents, guidewire or similar objects through it.

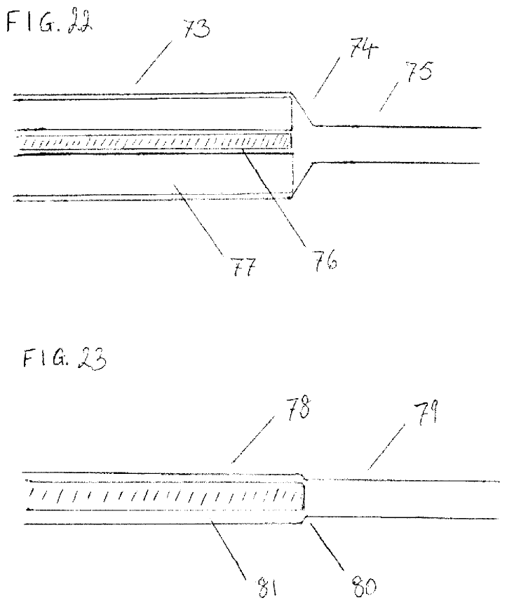

FIG. 22 is a longitudinal view of a hypotube which has a wider diameter portion 73 tapering in 74 to a narrower diameter portion 75. The purpose of this is to facilitate placement of a stent loader tube 77 into the wider portion. This allows for one person to place the stent 76 by pushing it with a stent inserter or guidewire into the narrow diameter part of the hypotube which is designed to fit in the bodily vessel. Once the stent pigtail has been pushed out the distal end of the hypotube, the hypotube can be withdrawn and the stent is placed. Other hypotube designs generally need two people to place the stent, one to push the stent loader tube tight against the hypotube and one to push the stent. This design also allows for the stent kit to be made from four parts, which makes it easier to use and easier to understand how to use it and reduces the possibility of error in the system with less working parts and no use of glue or other type of adhesive. A possible adaptation would be to form the proximal end of the hypotube into a luer lock or luer slip style connection to allow for tight contact with a syringe.

FIG. 23 is a longitudinal view of a hypotube with a wider diameter portion 78 tapering in 80 to a narrower diameter portion 79. This design could be used in larger diameter stents where there is more space available in the narrower diameter portion of the hypotube due to it being placed into wider vessels which can take a wider hypotube. The stent 81 would be pushed by a stent inserter or guidewire into the narrower diameter portion of the hypotube as in FIG. 22. The advantage of the wider diameter portion of the hypotube 78 is that it would facilitate placement of the stent into it. This type of pre-loaded design would be useful when the hypotube can be placed directly into the vessel without use of a guidewire which may be possible in pediatric usage. Use of a scope or direct vision may facilitate this method of placement which would be a very efficient method of placement, reducing the number of step in the procedure such as placement of a guidewire and the sheath or hypotube over the guidewire, removal of the guidewire and placement of the stent into the hypotube. A useful modification of this embodiment of the invention would be the incorporation of a cap at the proximal end of the hypotube to prevent the stent from falling out.

FIG. 24 is an iteration of the invention in which a tube is formed from a wire with braid or mesh extensions that can be pushed against each other to form a narrow low profile state to facilitate tracking along the vessel and that can be expanded by balloon or by removing a sheath or some similar method of expansion. The extensions form an incomplete tube in their high profile state. This iteration shows a longitudinal wire 82 attached to alternating braid extensions 83 in their high profile state.

FIG. 25 is an iteration of the invention in which a tube is formed by attaching a braid or a mesh or a coil 84 to a wire 85, said braid or mesh being expandable to be capable of being placed in a low profile state for faster and more flexible placement.

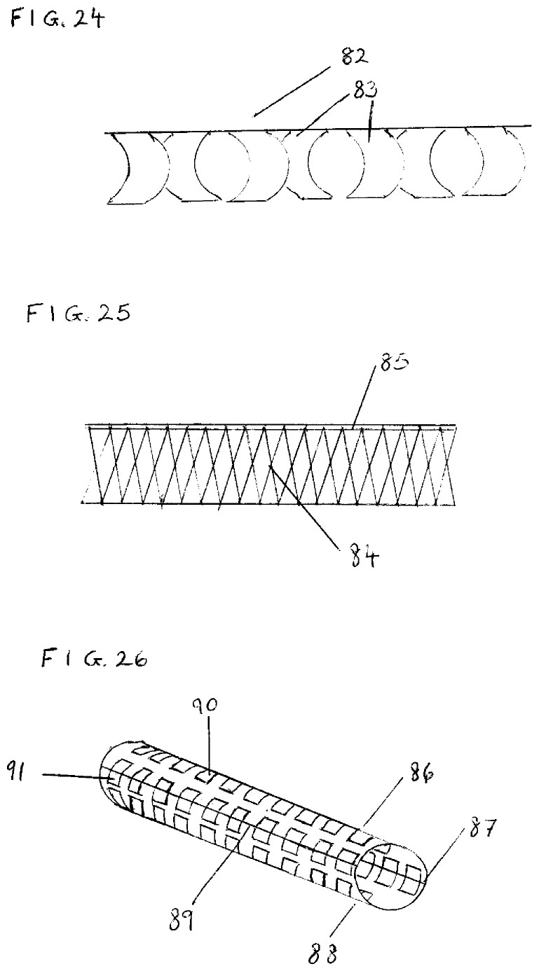

FIG. 26 is an iteration of the invention in which a tube is formed by a number of wires 86, 87, 88, 89 with braid or mesh or solid extensions 90, 91.

A further embodiment of the instant application are methods of installing or inserting a stent into a patient in need of a stent. The first method comprises using a motor, such as a single molecule electric motor, placed in the lumen or wall of a tube or on the distal end of a guidewire or stent itself. The motor could rotate a pulley-like device that when attached to the tube, guidewire or stent, draws the tube or guidewire or stent to a defined location in the body. Thereafter, a minimal force is applied to said tube, guidewire or stent to deliver said tube, guidewire or stent to the desired position in the patient wherein said minimal force causes minimal trauma to delicate vessels.

Of use to the tube in this invention is a method of inserting or installing a stent in a patient, which involves delivering the stent through a tube that is comprised of a proximal section having a wider inside diameter than the inside diameter of the distal section and using a stent inserter or introducer or a guidewire to push the stent or using gravity to move the stent from the wider inside diameter of the proximal section of the tube into a narrower section of the tube. A second tube may be optionally employed to hold the stent in the proximal section of the tube and optionally a cap may be placed on the proximal end of the tube. Further, a connection at the proximal end of the first tube, may be used to facilitate attachment of a syringe or a handle. This method, and the physical form of the tube used in it, allows very narrow medical devices, such as stents, endoscopes and guidewires, to be transitioned into a narrow distal section of a tube with ease. This is of particular value where shape memory is used in a part, an outer jacket is delicate, or there are concerns about damaging the distal end of a medical device when loading it into a narrow tube. It is not possible to flare a very narrow wall thickness in a tube to accommodate insertion of a medical device, hence the use of this method, which is to weld or attach a larger inner diameter tube to a narrower distal tube and pass the medical device into the narrow distal tube with ease.

In yet another embodiment of the invention, the invention of the instant application is directed to a guidewire, composed of a core wire having a distal and a proximal end and standard guidewire coatings, wherein the distal end of the core wire is attached to the inner surface of a series of hoops, coil or braid all of which having lumens, wherein said core wire does not pass through the center of the lumen of the hoops or coil or braid.

As a development of the means of placing a tube or a guidewire or a stent in a bodily vessel with a view to eliminating or reducing bodily trauma and maximizing surgical efficiency, which this patent application is generally concerned with, it may be worth investigating the possible benefits of a new method of placing same, whereby a surgical team would be less exposed to radiation from the use of fluoroscopy and would be less likely to suffer a health impact from same. This advancement would be to use a robotic system to place the device. The surgeon would make the initial incision and initial placement of the device in the vessel. It could then be tracked through the body using a sensitive pump or other pushing force that would advance the device smoothly and very slowly over very short distances through the vessel to an intended end point indicated by the surgeon using a GPS or other indicator on the body. The pump should have an automatic shut-off mechanism with an alerting system when there is any force transmitted back to it from the distal end of the device, due to encountering a stricture or an obstruction in the vessel or a difficult bend. Having established by imaging that pushing the device would not rupture the vessel, the surgeon would have to decide to increase the pushing force used or manually push through the obstruction using the tactile skills of a surgeon, depending on the issue encountered. It may be possible to program the machine to insert additional wiring into the tube or to change wiring in the tube to stiffen or make the tube more flexible which a surgeon would do by changing guidewires during an operation if an obstruction demanded it. It may also be possible to define the force necessary to rupture a vessel and ensure that the pump was not capable of that level of force, which could save lives. Advances in imaging and robotics may also allow for a machine to track a device using fluoroscopy, possibly from incision to final placement. This method of placing a device would reduce the time spent by surgeons in the theatre, since a number of operations could be monitored at the one time. It would reduce human error in medical device placement and it would also reduce surgical team exposure to radiation. The possibility of hardware and software error in any robotic system would obviously need to be tested very thoroughly, ideally mitigating the risk of either by ensuring the forces necessary to damage a vessel are not possible for the system.

In order to reduce the need to push a tube or guidewire through tortuous anatomy, which can cause trauma to the vessel, magnetic force, GPS or a similar system could possibly be used to attract a tube through a vessel to a given destination in the body without damaging the vessel walls. If pushing is necessary to move the tube or guidewire, a proximity sensor could be used to detect when the center of a tube or guidewire is against a wall. Technological advancement would be needed for some of these embodiments to function in a given space, but they would be welcome advancements for delicate, tortuous anatomy, such as can exist in a newborn child's diseased ureter, where breakage of a tube is not an option.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A tube having a distal and proximal end comprising a wire or rod or a plurality of longitudinal wires or rods or rods in a tube and wherein said tube is reinforced radially by a series of hoops or similarly shaped objects having a lumen or incomplete hoops, or by a braid, mesh, coil or the like, wherein the ability to apply longitudinal pressure to the tube without deformation is increased by the wire or rod;
further wherein the wire or rod adds structural rigidity to the tube; and
further wherein said tube is capable of being inserted into a patient.

2. The tube according to claim 1, wherein said wire or rod or plurality of longitudinal wires or rod or rods in an inner tube is/are ground down at the distal end of the wire or rod or plurality of longitudinal wires or rod or rods in an inner tube; or said wire or rod or plurality of longitudinal wires or rod or rods in an inner tube further comprise a coil, mesh, braid or a series of hoops at the distal end, wherein said coil, mesh, braid or series of hoops increase the flexibility of the distal end of said wire or rod or plurality of longitudinal wires or rod or rods in an inner tube.

3. The tube according to claim 1, wherein said wire or a plurality of longitudinal wires are capable of moving in and out of the tube.

4. The tube according to claim 1, further comprising wires inserted into the wall of the tube on alternating sides, such that at a given point, said tube may have a wire on one side and no wire on the opposite side, or alternatively, with a wire on one side and no wire on the opposite side and the reverse pattern after that, or alternatively, with a wire inserted into the wall of the tube followed by a gap followed by a wire inserted after the gap, wherein said alternating wire pattern provides increased flexibility at a given point or further comprising varying patterns of wires placed longitudinally at different points in the wall of the tube to provide varying flexibility on any axis.

5. The tube according to claim 1, further comprising a series of hoops or similarly shaped objects with lumens, or a coil, mesh, braid, expandable coil, expandable braid, expandable mesh or any combination thereof, attached longitudinally to a wire or a plurality of wires, or having a wire or a plurality of wires passed longitudinally through lumens that form part of the hoops or similarly shaped objects with lumens or coil or braid or mesh such that the longitudinal wires and latitudinal hoops or similarly shaped objects or coil or braid or mesh form a tube in terms of length and breadth.

6. The tube according to claim 1, further comprising an outer jacket having a lumen wherein said wire or plurality of wires are passed longitudinally through the lumen of the outer jacket or further comprising separate parts having lumens that are connected to the hoops or coil or braid or mesh wherein said wire or plurality of wires are passed longitudinally through the lumen of the separate parts connected to the hoops or coil or braid or mesh.

7. The tube according to claim 1, further comprising an inner liner, hydrophilic coating or other coating including coating with a drug, a marker band or other means of increasing the radiopacity of the tube or parts of the tube or any other means of adding materials to the tube or wherein said tube is formed into a spiral cup shape or a loop or a series of loops or a circular disc shape or a cup shape on top of an inverted cup shape at one or both ends.

8. The tube according to claim 1, further comprising a polymer or metallic cap that can substantially close the distal end of said tube, or a wire or wires which exert force on the distal end of the tube to substantially close said distal end, wherein when said polymer or metallic cap or wire is installed in said distal end of said tube, said cap can be uninstalled by the force of a stent, guidewire, endoscope or other similar item when said stent, guidewire, endoscope or other similar item is pushed by the user from the proximal end of the tube, through the length of the tube and out through the distal end of said tube.

9. The tube according to claim 1, wherein said tube is manufactured from a wire inserted into a series of hoops or similarly-shaped objects with lumens that are attached to a series of larger hoops or similarly-shaped objects with lumens with standard additions of outer jackets, inner liners, coatings and other standard tubing parts where desirable.

10. The tube according to claim 1, wherein said tube is manufactured from a wire attached to a series of triangular objects, or similarly-shaped objects or angled parts with a similar effect, with hoops or similarly shaped objects situated between the series of triangular objects or similarly shaped objects with standard additions of outer jackets, inner liners, coatings and other standard tubing parts.

11. A tube according to claim 1, wherein said tube is manufactured from a wire or a plurality of wires inserted into the lumen of a series of hoops or similarly-shaped objects with an inner liner placed into the lumen of the series of hoops or similarly-shaped objects, such that the wire or plurality of wires is between the inner liner and the hoops, and an outer jacket placed over the series of hoops or similarly-shaped objects.

12. A tube according to claim 1, wherein said tube is manufactured from a series of hoops or similarly shaped objects with a coil or a braid or a mesh or a series of gaps between the series of hoops with standard additions of outer jackets, inner liners, coatings and other standard tubing parts where desirable or wherein said tube is manufactured of a series of hoops or cut into a series of hoops with a spiral-cut or interrupted-cut pattern between the series of hoops with standard additions of outer jackets, inner liners, coatings and other standard tubing parts where desirable.

13. A tube according to claim 1, wherein said tube is cut into a series of incomplete hoops that are connected to each other by an uncut portion of the tube with standard additions of outer jackets, inner liners, coatings and other standard tubing parts where desirable.

14. A tube according to claim 1, wherein said tube is manufactured from a coil or a braid or a mesh attached longitudinally to a wire or a plurality of wires with standard additions of outer jackets, inner liners, coatings and other standard tubing parts where desirable.

15. A stent comprising a tube according to claim 1, wherein said tube is manufactured from a tube or metallic tube or a hypotube used to treat bodily vessel obstructions, said stent being formed by a laser cutting said tube, metallic tube or hypotube into a helix.

16. A guidewire for use in minimally invasive surgery comprising of a core wire having a distal and a proximal end, a series of hoops, coils, mesh, braids or a combination thereof wherein said hoops, coils, mesh or braid have an internal lumen, wherein the distal end of the core wire is attached to the inner surface of said hoops, coils, mesh or braids;
further wherein said core wire does not pass through the center of said lumen of said hoops, coils, mesh or braids and further comprising a protective external coating.

17. A method comprising use of a motor, such as a single molecule electric motor, placed in the lumen or wall of a tube or on the distal end of a guidewire or stent itself, said motor using linear or rotary force to move the tube or guidewire or stent to a defined location in the body.

18. A method of inserting or installing a stent in a patient by delivering the stent through a tube that is comprised of a proximal section having a wider inside diameter than the inside diameter of the distal section and using a stent inserter or introducer or a guidewire or gravity to move the stent from the wider inside diameter of the proximal section of the tube into a narrower section of the tube
wherein a cap may be placed on the proximal end of the tube;
wherein a second tube may be employed to hold the stent in the proximal section of the tube; and
further wherein a connection at the proximal end of the first tube may be used to facilitate attachment of a syringe.
